(19) United States
(12) Patent Application Publication
(10) Pub. No.: US 2008/0286215 A1
(43) Pub. Date: Nov. 20, 2008

(54) Method of Preventing Oral Staining

(76) Inventor: Rolf Valter Attstrom, Brinz (CH)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 12/091,617

(22) PCT Filed: Nov. 22, 2006
(86) PCT No.: PCT/GB2006/004357

§ 371 Date: Jul. 14, 2008

(30) Foreign Application Priority Data
Nov. 22, 2005 (GB) 0523743.3

(51) Int. Cl. A61K 8/49; A61Q 11/00

(52) U.S. Cl. 424/54

(57) ABSTRACT

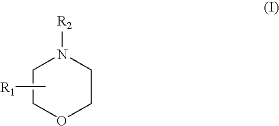

A cosmetic method of preventing or decreasing oral staining, comprising the step of exposing the oral cavity to a morpholino compound having the general formula (I) wherein R1 is a straight or branched alkyl group containing 8 to 16 carbon atoms at the 2- or 3-position of the morpholino ring, and R2 is a straight or branched alkyl group containing 2 to 10 carbon atoms, substituted with a hydroxy group except in the alpha-position, or a pharmaceutically acceptable salt thereof.

### FIELD OF THE INVENTION

The present invention relates to the prevention and removal of oral staining.

### BACKGROUND TO THE INVENTION

It is preferable to prevent unsightly staining of the oral cavity and it is particularly desirable to have non-stained, “white” teeth. A large number of de-staining and tooth whitening products and techniques exist. However, none of these are without their disadvantages; many require professional intervention and are expensive.

Common agents that cause discolouration of the oral cavity, in particular the teeth and tongue, include tea, coffee and cigarette smoke (i.e. tar staining). Chlorhexidine, an anti-microbial agent used to treat oral infections such as gingivitis, is also known to cause discolouration and staining, sometimes severe. Currently, chlorhexidine stains are only removed completely with the assistance of a professional hygienist (see for example Addy and Hunter, J Clin Periodontol. 1987 May; 14(5):267-73). Gerlach et al J Clin Dent. 2002; 13(1):10-4, indicate that removal of chlorhexidine and tea stains requires several weeks of stain removing activity.

Delmopinol is a morpholino compound that is useful as an anti-plaque agent. In some studies, dentists have noticed that prolonged use of delmopinol (to treat or prevent plaque) causes mild staining of the teeth, of some subjects.

A review of tooth discolouration and staining is given by Watts and Addy, B D J: Mar. 24, 2001, volume 190, no. 6, pages 309-316. It is clear that further methods and products that prevent and remove oral staining, in particular dental staining, are required. There is a particular need for an effective de-staining method that can be carried out without the need for professional intervention.

### SUMMARY OF THE INVENTION

The present invention is based on the surprising finding that a composition comprising Delmopinol (or a derivative or salt thereof) can reduce and prevent staining in the oral cavity. In particular, Delmopinol can whiten the teeth.

According to a first aspect of the invention, a cosmetic method of preventing or decreasing oral staining comprises the step of exposing the oral cavity to a morpholino compound having the general formula (I)

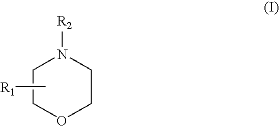

wherein R1 is a straight or branched alkyl group containing 8 to 16 carbon atoms at the 2- or 3-position of the morpholino ring, and R2 is a straight or branched alkyl group containing 2 to 10 carbon atoms, substituted with a hydroxy group except in the alpha-position, or a pharmaceutically acceptable salt thereof. A preferred salt is the hydrochloride (HCl) salt.

According to a second aspect of the invention, a kit for removing oral stains comprises a compound having the general formula (I) and instructions that the compound is to be used for oral stain removal.

According to a third aspect of the invention, a kit for whitening teeth comprises a compound having the general formula (I) and instructions that the compound is to be used for whitening teeth.

According to a fourth aspect of the invention, a kit comprises chlorhexidine and a compound having the general formula (I).

According to a fifth aspect of the invention, a compound having the general formula (I) is used in the manufacture of a composition for the removal of chlorhexidine staining.

### DESCRIPTION OF THE DRAWINGS

The invention is described with reference to the following drawings, wherein:

FIG. 1 panel B illustrates staining of the teeth caused when contacted over a seven-day period with Chlorhexidine 0.2% twice daily for one minute (panel A is a control showing seven days rinsing with Delmopinol 0.2% twice daily 10 ml for one minute);

FIG. 2 panel B illustrates staining of the tongue when contacted over a seven day period with Chlorhexidine 0.2% twice daily for one minute (panel A is a control showing seven days rinsing with Delmopinol 0.2% twice daily 10 ml for one minute); and

FIG. 3 illustrates the removal of Chlorhexidine stains seen in FIG. 1(B) by brushing the teeth for one minute with a toothbrush that has been soaked in 0.2% delmopinol.

### DETAILED DESCRIPTION OF THE INVENTION

Morpholino compounds with the general formula (I) are useful in removing and preventing staining in the oral cavity. A morpholino compound according to the invention has the general formula (I)

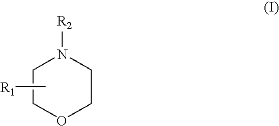

wherein R1 is a straight or branched alkyl group containing 8 to 16 carbon atoms at the 2- or 3-position of the morpholino ring, and R2 is a straight or branched alkyl group containing 2 to 10 carbon atoms, substituted with a hydroxy group except in the alpha-position, or a pharmaceutically acceptable salt thereof. In a preferred embodiment, the sum of the carbon atoms in the groups R1 and R2 of the morpholino compound is at least 10, preferably between 10 and 20. In a further preferred embodiment, the R2 group terminates with the hydroxy group.

The claimed morpholino compounds are known per se and can be manufactured by any known method, for example that disclosed in U.S. Pat. No. 5,082,653 and WO90/14342, which are incorporated herein by reference.

The preferred morpholino compound for use in the invention is 3-(4-propyl-heptyl)-4-(2-hydroxyethyl)morpholine, which is commonly known as Delmopinol (CAS No. 79874-76-3).

The morpholino compounds can be used in their free base form or as a pharmaceutically acceptable salt thereof. Examples of pharmaceutically acceptable salts are the salts of acids such as acetic acid, phosphoric acid, boric acid, hydrochloric acid, maleic acid, benzoic acid, citric acid, malic acid, oxalic acid, tartaric acid, succinic acid, glutaric acid, gentisic acid, valeric acid, gallic acid, beta-resorcyclic acid, acetyl salicylic acid, salicylic acid, perchloric acid, barbituric acid, sulfanilic acid, phytic acid, p-nitro benzoic acid, stearic acid, palmitic acid, oleic acid, myristic acid, lauric acid and the like. The most preferred salts are those of hydrochloric acid. A preferred compound is delmopinol hydrochloride (CAS No. 98092-92-3).

As used herein, the term “oral staining” refers to any unsightly discolouration of the oral cavity. In particular, it refers to staining of the teeth and tongue. The staining that is prevented and/or removed by a compound of formula (I) is caused by staining agents affecting the surfaces of the oral cavity, preferably the surface of the tongue and teeth, most preferably the surfaces of the teeth that are visible to others. This is commonly referred to as “extrinsic staining”. It is recognized in the art that extrinsic staining can be metallic or non-metallic (see, for example, Watts and Addy 2001, supra). Both metallic and non-metallic extrinsic staining are within the scope of the invention.

In a preferred embodiment, the compound of formula (I) is used to remove existing stains from the teeth.

The staining can be caused by any staining agent. A number of foods and drinks can cause dental staining including, tea, coffee, cola and red wine. These food and drink based staining agents are referred to herein as “dietary” staining agents. Cigarette and pipe smoke (i.e. tar in the water phase from the smoking tobacco) is also known to cause staining. Further, smokeless tobacco also causes staining of the teeth and tongue but to a lesser degree than smoking tobacco. The anti-bacterial agent Chlorhexidine, which is used to treat oral infections, also causes staining of the teeth. Compounds of the formula (I) can remove stains caused by these agents (amongst others), and prevent further staining.

It should be understood that the term “oral staining” includes staining of teeth, i.e. dental staining. The compound of formula (I) can therefore be used to prevent and remove dental staining. The removal of (dark) stains from the teeth, to give them a “whiter” appearance, is referred to herein as “whitening” of the teeth.

The oral cavity is exposed to a compound of formula (I). The compound of formula (I) may be brought into contact with the oral cavity in a conventional way, in any suitable form or amount that achieves the desired effect, i.e. reduction or prevention of staining in the oral cavity. Preferably, the compound of formula (I) is in the form of an aqueous mouthwash, toothpaste, gel, gum, dentifrice or other similar preparation that will be apparent to the skilled person. In a preferred embodiment, mechanical agitation, preferably brushing the teeth, tongue and gums, is performed simultaneously with or shortly, preferably immediately, after contacting the oral cavity with a compound of formula (I). Most preferably, the compound of formula (I) is applied as an aqueous mouthwash at the start of any regular (e.g. daily) oral health routine, such as before brushing the teeth. In a further preferred embodiment, the oral cavity is contacted with a compound of formula (I) shortly, preferably immediately, before contacting the oral cavity with a further de-staining agent.

The compound of formula (I) can be added to an existing toothpaste, gum, gel or mouthwash formulation. The compound of formula (I) may be added in combination with at least one other agent that is known to remove/prevent staining. Preferably, the additional agent comprises a peroxide-containing whitener or bleaching agent. The combination of a compound of formula (I) and another agent that is known to remove/prevent staining demonstrates a surprising synergy that allows stains to be removed, i.e. teeth to be whitened, more effectively than when each of the agents is used separately.

Alternatively, the compound of formula (I) is the only active de-staining ingredient in the preparation with which the oral cavity is contacted.

In a further preferred embodiment, the compound of formula (I) is added to the oral cavity in combination with at least one abrasive agent such as sodium bicarbonate, calcium phosphates, calcium carbonate and silica.

In a preferred embodiment, contacting the oral cavity with a compound of formula (I) is not the final step in a regular oral health regime. In a preferred embodiment, the compound of formula (I) is contacted with the oral cavity prior to a brushing or swilling step. It has been reported that delmopinol can, in some subjects, cause mild staining. Ensuring that the application of the compound of formula (I) is not the final step in a regular oral health regime reduces or prevents the possibility that the compound of formula (I) will itself cause staining. An example of a suitable regime is swilling or brushing with a compound of formula (I) followed by brushing the teeth with toothpaste, gel or dentifrice. When chlorhexidine is used as part of the oral health regime, it is preferred that the compound of formula (I) is contacted with the oral cavity after the chlorhexidine treatment, but, preferably, before brushing the teeth.

The compound of formula (I) can be used at any suitable concentration, which will be apparent to one skilled in the art. In a preferred embodiment, the concentration of the compound of formula (I) is between 0.01% (w/v) and 10% (w/v), preferably from 0.1% (w/v) to 5% (w/v), such as 0.2% (w/v), 1% (w/v), 2% (w/v) or 3% (w/v).

A kit or pack comprising a compound of formula (I) and instructions for use in removal or prevention of oral stains, together with instructions directing the use of the compound for removal or prevention of stains, is included in the invention. Preferably, the kit is intended to whiten the teeth and the instructions recite this. In a further preferred embodiment, the kit or pack is intended to remove chlorhexidine stains. In this embodiment, the kit may contain chlorhexidine together with a compound of formula (I), and instructions for using the compound of formula (I) is intended to remove or prevent staining caused by the chlorhexidine. Preferably, the chlorhexidine and compound of formula (I) are packaged separately within the kit or pack.

The invention is further described with reference to the following non-limiting example.

### EXAMPLE

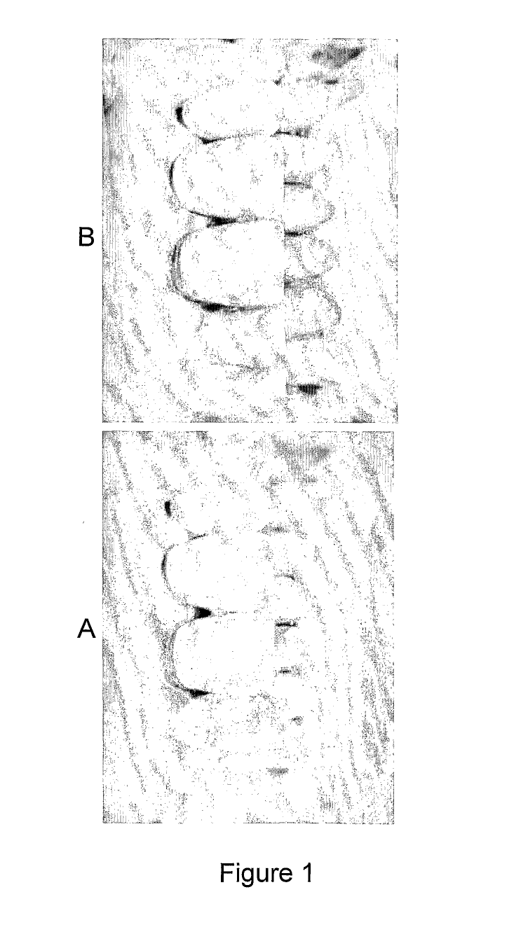

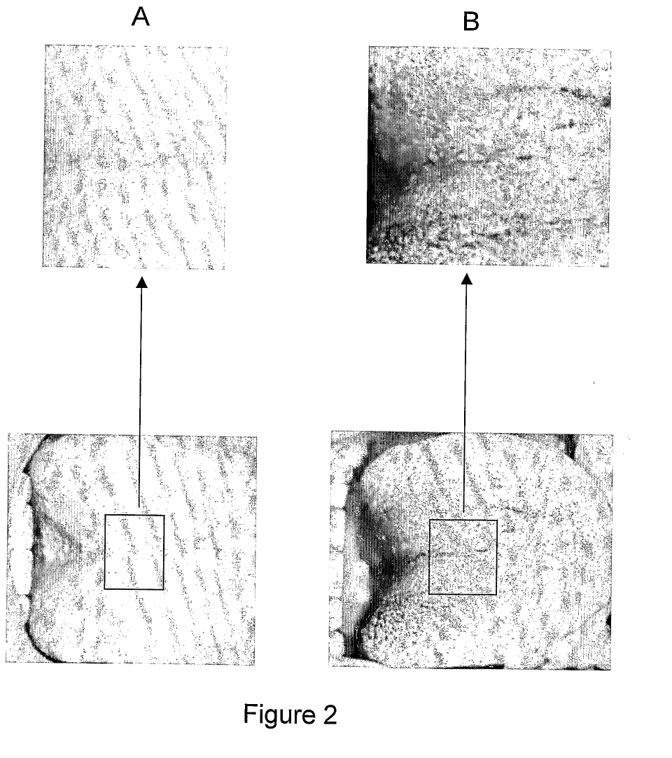

A subject swilled their mouth twice daily with a 0.2% chlorhexidine mouthwash for seven days. The characteristic staining of the teeth that resulted from this chlorhexidine use is illustrated in FIG. 1B; the tongue was also stained by this seven day use of chlorhexidine, as illustrated in FIG. 2B.

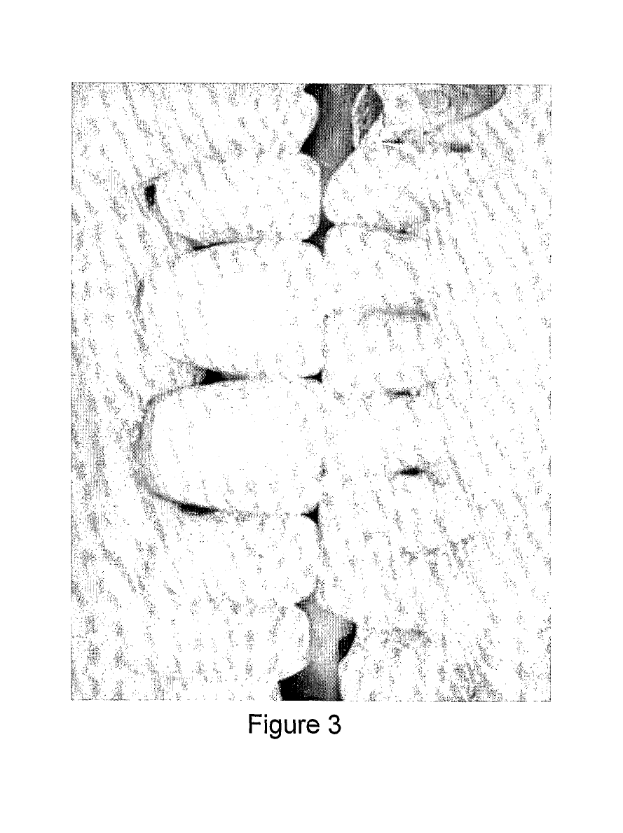

The subject with chlorhexidine staining (after the seven day usage of chlorhexidine) brushed their teeth once, for one minute, using a toothbrush soaked in a 0.2% delmopinol mouthwash. The results of this are shown in FIG. 3. It is clear that this single brushing of the teeth with delmopinol has removed the staining of the teeth seen in FIG. 1B.

As a separate control, the subject swilled their mouth twice daily with a 0.2% delmopinol mouthwash for seven days. No staining was observed on the teeth or tongue, as illustrated in FIGS. 1A and 2A, respectively.

1. A cosmetic method of preventing or decreasing oral staining, comprising the step of exposing the oral cavity to a morpholino compound having the general formula (1) 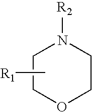
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or a pharmaceutically acceptable salt thereof.

2. The cosmetic method according to claim 1, wherein the sum of the carbon atoms in the groups R₁ and R₂ of the morpholino compound is at least 10.

3. The cosmetic method according to claim 1, wherein R₂-of the morpholino compound terminates with the hydroxy group.

4. The cosmetic method according to claim 1, wherein the morpholino compound is 3-(4-propyl-heptyl)-4-(2-hydroxyethyl) morpholine.

5. The cosmetic method according to claim 1, wherein the teeth are whitened.

6. The cosmetic method according to claim 1, wherein the staining is from chlorhexidine.

7. The method according to claim 1, wherein the staining is from nicotine.

8. The method according to claim 1, wherein the staining is from dietary stains.

9. The method according to claim 1, additionally comprising brushing the oral cavity.

10. (canceled)

11. The method according to claim 1, wherein the compound of formula (I) is present in a mouthwash, toothpaste, gum or gel.

12. A kit for removing oral stains or for whitening teeth, comprising a compound having the general formula (1) 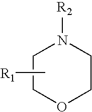
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or a pharmaceutically acceptable salt thereof, and instructions that the compound is to be used for oral stain removal or for whitening teeth.

13. (canceled)

14. The kit according to claim 12 comprising chlorhexidine and a compound having the general formula (1) 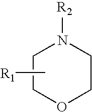
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or a pharmaceutically acceptable salt thereof.

15. A method for the manufacture of a composition for the removal of chlorhexidine staining wherein said method comprises the use of a compound having the general formula (1) 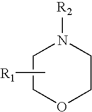
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or a pharmaceutically acceptable salt thereof.

16. The method, according to claim 15, wherein the composition is a toothpaste, mouthwash, gum or gel.

17. A method for whitening teeth wherein said method comprises the use of a compound having the general formula (1) 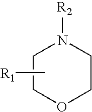
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or a pharmaceutically acceptable salt thereof.

18. The method, according to claim 17, wherein the compound of formula (I) is present in a mouthwash, toothpaste, gum or gel.
